(19) United States
(12) Patent Application Publication
(10) Pub. No.: US 2025/0161537 A1
(43) Pub. Date: May 22, 2025

(54) IN-SITU INJECTABLE HYDROGEL ROD USING CROSSLINKED HYDROGEL, METHOD FOR MANUFACTURING SAME, AND BIOMEDICAL USE THEREOF

(71) Applicants: AJOU UNIVERSITY INDUSTRY-ACADEMIC COOPERATION FOUNDATION, Suwon-si, Gyeonggi-do (KR); SEOUL NATIONAL UNIVERSITY HOSPITAL, Seoul (KR)

(72) Inventors: Ki Dong PARK, Seoul (KR); Si Min LEE, Suwon-si Gyeonggi-do (KR); Se Joon WOO, Seongnam-si Gyeonggi-do (KR); Hye Kyoung HONG, Seongnam-si Gyeonggi-do (KR)

(73) Assignees: AJOU UNIVERSITY INDUSTRY-ACADEMIC COOPERATION FOUNDATION, Suwon-si, Gyeonggi-do (KR); SEOUL NATIONAL UNIVERSITY HOSPITAL, Seoul (KR)

(21) Appl. No.: 18/836,391

(22) PCT Filed: Nov. 4, 2022
(86) PCT No.: PCT/KR2022/017200

§ 371 Date: Aug. 7, 2024

(30) Foreign Application Priority Data
Feb. 7, 2022 (KR) 10-2022-0015609

(51) Int. Cl. A61L 27/52; A61L 27/02; A61L 27/14; A61L 27/54

(52) U.S. Cl. CPC A61L 27/52; A61L 27/025; A61L 27/14; A61L 27/54; A61L 2300/11; A61L 2300/254; A61L 2300/602; A61L 2400/06

(57) ABSTRACT

The present invention relates to an injectable hydrogel rod using a crosslinked hydrogel, a method for manufacturing same, and biomedical use thereof. According to the present invention, by adjusting the contents of a polymer, an enzyme and hydrogen peroxide to a certain ratio to manufacture an injectable hydrogel rod, the initial release amount of a drug injected into the hydrogel rod is controlled, and thus, compared to existing hydrogel-based injectable implants, the hydrogel rod exhibits sustained release above an effective concentration for a long period of time and greatly reduces elimination into surrounding tissues, which may result in an increased therapeutic effect and remarkably reduced side effects.

### TECHNICAL FIELD

The present disclosure relates to an injectable hydrogel rod using a crosslinked hydrogel, a manufacturing method thereof, and a biomedical use thereof.

### BACKGROUND ART

Hydrogel, a jelly-like substance through physicochemical crosslinking of polymers dissolved in water, is used as a support for tissue engineering or drug delivery because it is able to easily absorb water due to its excellent hydrophilicity while its strength and shape are easily adjustable. In addition, due to outstanding biocompatibility, biodegradability, and minimally invasive properties to regulate drug release and achieve phase transition after in vivo injection, hydrogels are being actively researched as an alternative to methods showing low efficiency in the in vivo drug delivery. As a drug delivery carrier, its main purpose is to eliminate the need for frequent injections, which may cause side effects and discomfort such as pain through sustained release of drugs above the effective concentration for a long period of time at a site of the disease. Typically, in the case of wet age-related macular degeneration among ophthalmic diseases, an anti-VEGF agent, which is a protein drug, should be injected intraocularly for treatment, but the intraocular half-life is less than two (2) weeks, and the duration of the drug efficacy is short, lasting 1-2 months. Therefore, in order to derive the maximum effectiveness of the anti-VEGF agent, intraocular injection is required almost every month, which not only causes a great deal of discomfort and side effects to patients, but also poses a substantial economic burden, such that injectable hydrogels are being developed as a promising intraocular protein drug delivery carrier.

However, injectable hydrogels show limitations in controlling a rate of drug release in diseases where long-term maintenance of drug effectiveness is required, since an excess amount of drug is released at an initial stage before phase transition occurs after injection. In order to control the rate of drug release, it is necessary to inject hydrogel that had undergone crosslinking and phase transition, but the crosslinked hydrogel is solidified and in an irregular form which cannot be injected into the body. Accordingly, the present inventors have newly developed a long-term protein drug release-controlled injectable implant (hydrogel rod) using a crosslinked hydrogel.

### DISCLOSURE OF THE INVENTION

### Technical Goals

The present disclosure relates to an injectable hydrogel rod for control of release of a protein drug using a crosslinked hydrogel, a manufacturing method thereof, and a use thereof, and an object of the present disclosure is to provide an injectable hydrogel rod which exhibits sustained release properties by controlling the contents of a polymer, an enzyme, and hydrogen peroxide.

### Technical Solutions

In order to achieve the above object, the present disclosure provides an injectable hydrogel rod including a rod-shaped hydrogel matrix which includes a polymer substituted with phenol derivatives and in which the phenol derivatives introduced into side chains of the polymer are bonded to each other and crosslinked; and a drug loaded in the hydrogel matrix.

In addition, the present disclosure provides an injectable implant material including the injectable hydrogel rod described above.

In addition, the present disclosure provides a drug delivery carrier including the injectable hydrogel rod as described above.

In addition, the present disclosure provides a method of manufacturing an injectable hydrogel rod, including preparing a mold having a rod-shaped hole; preparing a lysate in which a polymer substituted with a phenol derivative, a drug, and an enzyme are dissolved; injecting the lysate and hydrogen peroxide sequentially into the hole provided in the mold and then crosslinking hydrogel; separating the crosslinked hydrogel from the mold; and freeze-drying the separated hydrogel to manufacture a hydrogel rod,

wherein a mixing ratio of the polymer substituted with the phenol derivative, the enzyme, and the hydrogen peroxide is 10:0.1˜0.5:0.1˜0.5 in a volume ratio.

### Advantageous Effects

According to the present disclosure, by manufacturing an injectable hydrogel rod by controlling the contents of a polymer, an enzyme, and hydrogen peroxide in a certain ratio, it is possible to control an initial burst release amount of drug released from the hydrogel rod to ensure sustained release above a concentration of drug efficacy for a long period of time compared to existing in situ forming hydrogels, and remarkably decrease elimination to the surrounding tissues, thereby significantly reducing side effects.

### BRIEF DESCRIPTION OF DRAWINGS

FIG. 1 shows (a) a schematic diagram illustrating a manufacturing method of an injectable hydrogel rod (GPT hydrogel rod) according to the present disclosure, (b) an image of a shape of the injectable hydrogel rod manufactured in the present disclosure, and (c) a schematic diagram of fabricating an injector of the injectable hydrogel rod manufactured in the present disclosure.

FIG. 2 shows (a) a table showing a manufacture condition of an injectable hydrogel rod according to the present disclosure, and (b) a graph that evaluated mechanical strength in accordance with the control of a degree of crosslinking of the injectable hydrogel rod of the present disclosure.

FIG. 3 shows (a) a graph of a time-specific drug release amount of an injectable hydrogel rod according to the present disclosure and (b) a graph showing a cumulative release behavior until 120 days.

FIG. 4 shows (a) a schematic diagram of an in situ forming hydrogel of Comparative Example, (b) a schematic diagram of drug release incubation of an injectable hydrogel rod of Example, and (c) a graph of a drug release behavior of in situ forming hydrogel and injectable hydrogel rod.

FIG. 5 shows (a) graphs showing growth inhibition of HUVEC by released drug from an injectable hydrogel of Example and an in situ forming hydrogel of Comparative Example through WST-1 assay, and (b) scanning electron microscope (SEM) images showing cytocompatibility and growth inhibition of HUVEC through Live/Dead assay.

FIG. 6 is a schematic diagram of an intraocular injection plan for in vivo pharmacokinetic evaluation of an injectable hydrogel of Example compared to an in situ forming hydrogel of Comparative Example according to the present disclosure.

FIG. 7 shows (a-c) graphs showing pharmacokinetic results of each tissue in the eye of an injectable hydrogel rod of Example and an in situ forming hydrogel of Comparative Example, and (d) a table showing drug concentration of each tissue.

FIG. 8 is intraocular ultrasound images for each time point after intraocular injection of an injectable hydrogel rod according to the present disclosure.

### BEST MODE FOR CARRYING OUT THE INVENTION

Hereinafter, in order to explain the present disclosure in more detail, preferred example embodiments according to the present disclosure will be described in more detail with reference to the attached drawings. However, the present disclosure is not limited to the example embodiments described herein but may be implemented in other forms.

The present inventors have completed the present disclosure through the design and effectiveness evaluation on a manufacturing method of a long-term protein drug release-controlled injectable hydrogel rod using a crosslinked hydrogel.

The present disclosure provides an injectable hydrogel rod which includes a rod-shaped hydrogel matrix which includes a polymer substituted with phenol derivatives and in which the phenol derivatives introduced into side chains of the polymer are bonded to each other and crosslinked; and a drug loaded in the hydrogel matrix.

The hydrogel matrix may have a degree of crosslinking of 30% to 80%, 40% to 70%, or 45% to 60%. With the degree of crosslinking as described above, the hydrogel rod may exhibit sustained release properties for more than 3 or 4 months by controlling an initial release rate to 10% or less, 7% or less, or 5% or less.

The rod-shaped hydrogel matrix may be 0.5 to 0.7 mm, 0.7 to 0.9 mm, or 0.9 to 1.2 mm in diameter.

In addition, the rod-shaped hydrogel matrix may be 4 to 6 mm, 6 to 8 mm, or 8 to 10 mm in length.

The polymer may be one or more polymers selected from the group consisting of gelatin, chitosan, heparin, cellulose, dextran, dextran sulfate, chondroitin sulfate, keratan sulfate, dermatan sulfate, alginate, collagen, albumin, fibronectin, laminin, elastin, vitronectin, hyaluronic acid, fibrinogen, and polybranched-polymers.

The phenol derivative may be one or more selected from the group consisting of hydroxyphenyl propionic acid, 4-hydroxyphenyl acetic acid, tyrosine, tyramine, tetronic tyramine, and PEG-tyramine.

The drug may be one or more drugs selected from intraocularly injectable protein therapeutic agents, chemotherapeutic drugs, low molecular weight therapeutic agents, genes, cells, or cell derivatives. Specifically, protein therapeutic agents may consist of anti-vascular endothelial growth factors used in macular degeneration and diabetic retinopathy, in other words, anti-VEGF ranibizumab, aflibercept, bevacizumab, brolucizumab, and other protein drugs used in diseases such as rituximab, adalimumab, or infliximab; the chemotherapeutic drug may consist of dexamethasone, triamcinolone, ganciclovir, methotrexate, or vancomycin; the low molecular weight therapeutic agent may consist of sugars, lipids, amino acids, fatty acids, phenolic compounds, or alkaloids; the gene may consist of siRNAs (anti-VEGF); and the cell may consist of retinal and intraocular cells such as RPE and photoreceptors, and stem cells. The following example embodiments using bevacizumab as a drug are described, but the present disclosure is not limited to these types of drugs and may include protein therapeutic agents, chemotherapeutic drugs, low molecular weight therapeutic agents, genes or cells of any ophthalmic and various diseases.

The hydrogel may form a rod-shaped hydrogel matrix by controlling concentration of the polymer, an enzyme, and hydrogen peroxide.

Specifically, the polymer substituted with the phenol derivatives, the enzyme, and the hydrogen peroxide may be mixed in a volume ratio of 10:0.1˜0.5:0.1˜0.5 to form a rod-shaped hydrogel matrix.

In addition, the present disclosure provides an injectable implant material including the injectable hydrogel rod as described above. Specifically, the injectable implant material may be an intraocularly injectable implant material.

The implant material may be applied to any one selected from the group consisting of intraocular and extraocular spaces in the body, subcutaneous tissues, and other interiors of organs.

In addition, the present disclosure provides a drug delivery carrier including the injectable hydrogel rod as described above.

In addition, the present disclosure provides a method of manufacturing an injectable hydrogel rod, including preparing a mold having a rod-shaped hole; preparing a lysate in which a polymer substituted with a phenol derivative, a drug, and an enzyme are dissolved; injecting the lysate and hydrogen peroxide sequentially into the hole provided in the mold and then crosslinking hydrogel; separating the crosslinked hydrogel from the mold; and freeze-drying the separated hydrogel to manufacture a hydrogel rod,

wherein a mixing ratio of the polymer substituted with the phenol derivative, the enzyme, and the hydrogen peroxide is 10:0.1˜0.5:0.1˜0.5 in a volume ratio.

In the preparing of the mold, the mold may be prepared to have a rod-shaped hole inside one type of mold selected from the group consisting of gelatin, metal, and Teflon.

The rod-shaped hole may have a diameter of 0.5 to 1.2 mm and a length of 4 to 10 mm.

Specifically, the preparing of the mold may include preparing a gelatin mold by pouring a gelatin solution on the outside of a needle and then hardening at a temperature of 1 to 10° C.

The needle may have a diameter of 0.5 to 1.2 mm and a length of 4 to 10 mm.

The gelatin solution may include 5 to 20 wt % or 5 to 15 wt % of gelatin.

In the crosslinking of the hydrogel, one or more phenol groups introduced into a side chain of the polymer may be bonded to each other and crosslinked through an enzymatic crosslinking reaction of the enzyme and the hydrogen peroxide. Specifically, it is possible to form a rod-shaped hydrogel through the crosslinking of hydrogel.

The polymer may be any one or more polymers selected from the group consisting of gelatin, chitosan, heparin, cellulose, dextran, dextran sulfate, chondroitin sulfate, keratan sulfate, dermatan sulfate, alginate, collagen, albumin, fibronectin, laminin, elastin, vitronectin, hyaluronic acid, fibrinogen, and polybranched-polymers.

The enzyme includes one or more selected from the group consisting of horseradish peroxidase, glutathione peroxidase, haloperoxidase, myeloperoxidase, catalase, hemoprotein, peroxide, peroxiredoxin, animal heme-dependent peroxidase, thyroid peroxidase, vanadium bromoperoxidase, lactoperoxidase, tyrosinase, and catechol oxidase.

A content of the polymer may be 10 to 30 wt % or 15 to 25 wt %.

A content of the hydrogen peroxide may be 0.02 to 0.08 wt %, 0.03 to 0.07 wt %, or 0.035 to 0.06 wt %.

A concentration of the enzyme may be 0.001 to 0.005 mg/ml or 0.002 to 0.004 mg/ml.

By mixing the polymer, enzyme, and hydrogen peroxide as described above, the injectable hydrogel rod manufactured according to the present disclosure exhibits a degree of crosslinking of 20% to 100%, 30% to 80%, 40% to 70%, or 45% to 60%, and thus the hydrogel rod may exhibit sustained release properties for more than three (3) months by controlling the initial release rate to 10% or less, 7% or less, or 5% or less.

In the separating from the mold, the rod-shaped hydrogel may be separated from one type of molds selected from the group consisting of gelatin, metal, and Teflon.

Specifically, in the separating from the mold, the rod-shaped hydrogel may be separated by dissolving the gelatin at a temperature of 30° C. to 40° C. to remove the gelatin mold.

Alternatively, in the separating from the mold, the hydrogel rod in the mold may be separated by pushing with a rod-shaped mold that is made of metal or Teflon materials.

The freeze-drying may be performed at a temperature of −70 to −85° C. for 12 to 24 hours to manufacture a hydrogel rod.

### MODES FOR CARRYING OUT THE INVENTION

Hereinafter, the present disclosure will be described in more detail through examples to help understanding of the present disclosure. However, examples below are merely intended to illustrate the present invention, and the scope of the present disclosure is not limited to the following examples. Examples of the present disclosure are provided to more completely explain the present disclosure to those skilled in the art.

### Examples and Comparative Examples

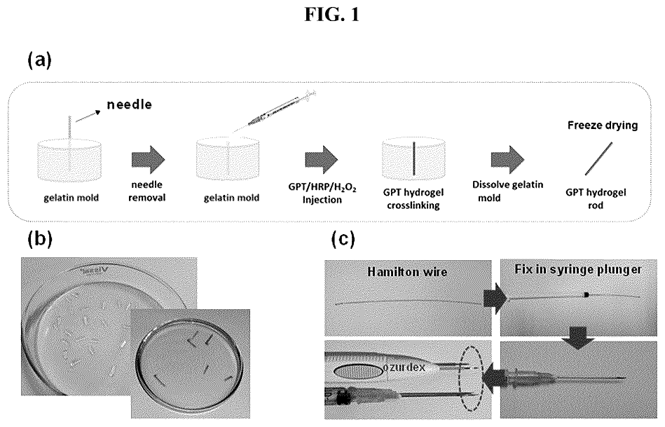

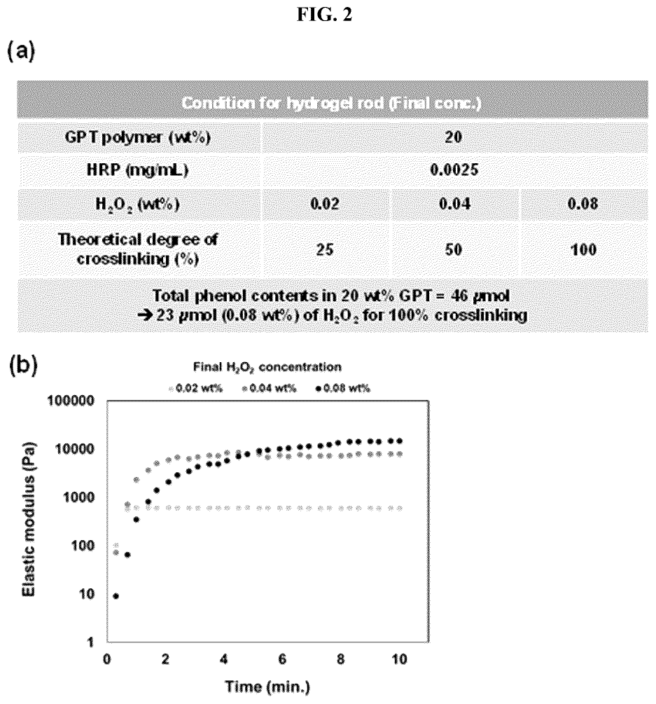

Temperature-sensitive gelatin was used as a mold to manufacture a rod-shaped drug delivery carrier of injectable gelatin-PEG-tyramine (GPT) hydrogel ((a) of FIG. 1). After fixing a needle in the well of a 48-well plate, a gelatin solution at 37° C. (10 wt %) was added to the outside of the needle, and then a solid gelatin mold was formed through refrigeration at 4° C. After freeze-drying 1 mL of bevacizumab at a concentration of 25 mg/mL, it was dissolved in 250 μL of GPT polymer solution to prepare a GPT polymer solution mixed with bevacizumab drug at a concentration of 100 mg/mL. Thereamong, 15 μL of GPT polymer solution loaded with 1500 g of bevacizumab:HRP:H₂O₂ were mixed in a ratio of 9:0.5:0.5 respectively to be used to manufacture a hydrogel rod in a total volume of 16.67 L. After removing the needle, enzymatic crosslinking was performed by adding the GPT hydrogel solution mixed with bevacizumab drug inside the mold. 1500 g of bevacizumab was loaded, and the concentration of GPT polymer, HRP, and H₂O₂ was used as shown in Table 1 below. Phase transition was carried out in the mold using the enzymatic crosslinking method using HRP/H₂O₂. After 2 hours of incubation, the crosslinked GPT hydrogel rod was obtained by dissolving the gelatin mold at 37° C. (FIG. 1 (b)), followed by freeze-drying and storage. To develop an injector for injecting the hydrogel rod, a Hamilton syringe wire was used by fixing onto a plunger of 1 mL syringe. The manufactured injector may be applied in a manner similar to the commercial product Ozurdex (FIG. 1 (c)).

TABLE 1

| | Final | Final concentration for | |
|---|---|---|---|
| | concentration of | crosslinking condition | |
| Category | polymer (wt %) | HRP (mg/mL) | H₂O₂ (wt %) |
| Example 1 | 20 | 0.0025 | 0.02, 0.04, 0.08 |
| Comparative | 3 | 0.0005~0.0025 | 0.0075, 0.0085, |
| Example 1 | | | 0.00925, 0.01 |
| Comparative | 5 | 0.00625~0.1 | 0.0025, 0.003, |
| Example 2 | | | 0.004, 0.0045, |
| | | | 0.00625 |

### Experimental Example 1—Mechanical Properties

As shown in FIG. 1, if hydrogel rods are manufactured in the compositions of Example 1 and Comparative Examples 1 and 2, the rod shape is not retained after acquisition by freeze-drying due to low concentrations of polymers and a lack of polymer chains upon rod fabrication in Comparative Examples 1 and 2, whereas, as shown in (b) of FIG. 1, in the case of Example 1, it is noticeable that the hydrogel rod was fabricated under optimal conditions.

In order to identify the mechanical properties in accordance with the control of the degree of crosslinking of the injectable hydrogel rod of the present disclosure, in Example 1, the final concentration of H₂O₂ used for crosslinking was adjusted to 0.02, 0.04, and 0.08 wt % as shown in (a) of FIG. 2 in an attempt to control the theoretical degree of crosslinking to 25, 50, and 100%. After injecting 300 μL of GPT hydrogel into the plate of the rheometer for each condition before being crosslinked, as a result of measuring the changes in the mechanical properties over time as the crosslinking progresses through the rheometer, the mechanical properties of the matrix increased to 0.6, 8.1, and 15 kPa, respectively, according to the increase in the theoretical degree of crosslinking, as shown in (b) of FIG. 2.

### Experimental Example 2—Evaluation of Drug Release Control by Diversifying Crosslinking Conditions of Hydrogel Rods

### 2-1. Evaluation of Drug Release Control Following Control of Degree of Crosslinking

Following evaluation of the control of physical properties upon control of a degree of crosslinking of the injectable hydrogel rod, the evaluation of the control of bevacizumab release was carried out. While the hydrogel rod formed by the degree of crosslinking was incubated in DPBS (37° C., 100 rpm), a DPBS sample including bevacizumab released at the specific time point for 120 days was obtained, frozen, and stored, followed by addition of a new DPBS solution. The obtained samples were subjected to quantitative analysis using the enzyme linked immunosorbent assay (ELISA) technique, which enables specific quantification of bevacizumab, an anti-VEGF.

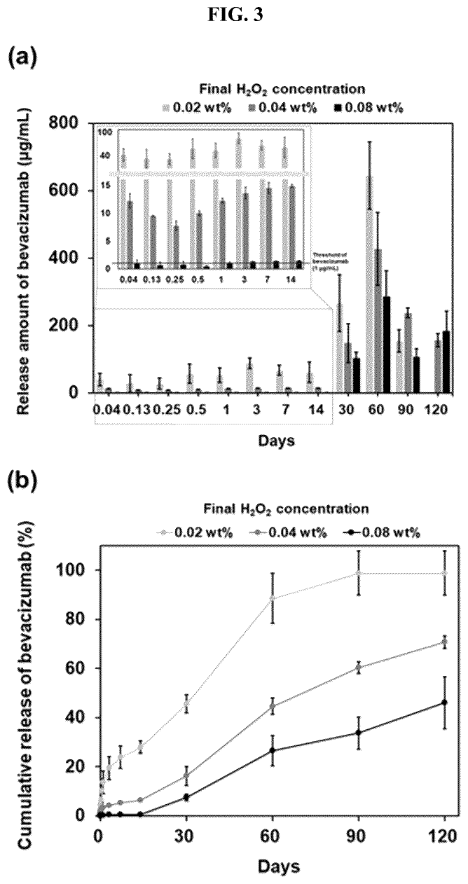

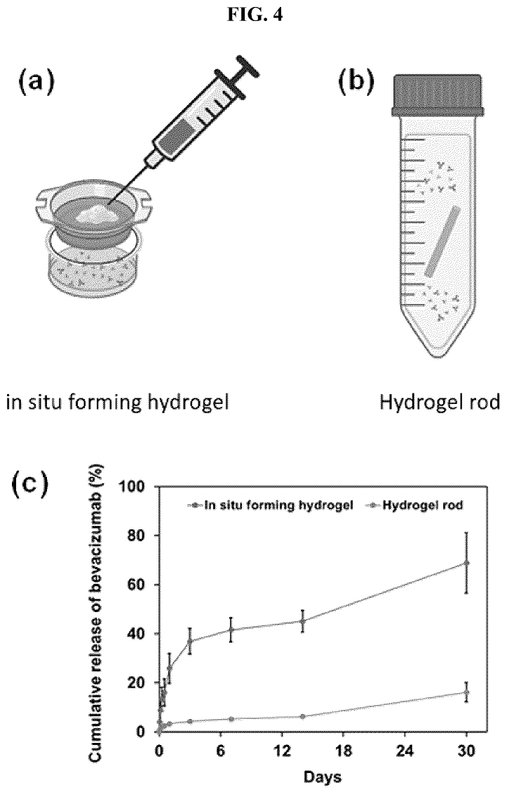

As shown in (a) of FIG. 3, the result of in vitro drug release of the hydrogel rod for 120 days in H₂O₂ concentrations of 0.02, 0.04, and 0.08 wt % with the degree of crosslinking adjustable showed a decrease in the drug release amount at each time with the increase in the degree of crosslinking. Specifically, while a threshold for drug efficacy of bevacizumab is known to be approximately 1 g/mL (IOVS, October 2008, Vol. 49, No. 10), by the initial 14 days, 0.02 wt % condition resulted in an excess amount of release beyond the threshold, 0.08 wt % condition resulted in a trace amount of release that falls short of the threshold, and 0.04 wt % condition resulted in the release in the vicinity of 10 g/mL above the threshold, followed by the control of the drug release amount according to the degree of crosslinking.

Referring to (b) of FIG. 3, as a result of the cumulative release up to 120 days, the initial burst release and release rate decreased in proportion to the degree of crosslinking, and the most suitable release pattern was shown under the 0.04 wt % condition.

### 2-2. Comparative Evaluation on Drug Release in Examples and Comparative Examples

The comparison was made on drug release behaviors of the hydrogel rod (pre-crosslinked hydrogel rod) of Examples and the in situ forming hydrogel of Comparative Example. Polymers under the same condition (GPT 20 wt %) and crosslinking conditions (H₂O₂ 0.04 wt %) were used, and 1500 g of bevacizumab was loaded, followed by evaluation of the release. Referring to (a) and (b) of FIG. 4, the in situ forming hydrogel of Comparative Example was injected into DPBS (37° C., 100 rpm) using a well plate insert just before crosslinking by mimicking a minimally invasive injection environment in vivo, and the hydrogel rod was incubated in DPBS (37° C., 100 rpm). Referring to (c) of FIG. 4, as a result of evaluation via ELISA after 30 days of cumulative release, the in situ forming hydrogel of Comparative Example showed cumulative release of more than 60% up to 30 days after the initial excess release of about 40% until 3 days. It can be seen that crosslinking may occur that falls short of the target degree of crosslinking due to dilution with medium solutions upon injection of the in situ forming hydrogel, thereby leading to the rapid spread of drug to surrounding media. On the other hand, as the hydrogel rod of Example was used in a crosslinked state, the initial release was minimized to less than 5%, resulting in about 20% cumulative release until 30 days. These results show that hydrogel rods have advantages in terms of initial release inhibition and long-term sustained release.

As a result of conducting comparative evaluation with the in situ forming hydrogel that is not in the form of a rod form of Comparative Example as above, the in situ forming hydrogel of Comparative Example is a substance that undergoes phase transition after minimally invasive injection and also quickly releases the loaded drug at an initial stage, but the hydrogel rod of the present disclosure may enable minimally invasive injection to be applied in a crosslinked form to a pre-targeted degree, such that it was determined that the loaded drug is prevented from being released quickly at an early stage and exhibits sustained release for more than four (4) months.

### Experimental Example 3—Evaluation of Long-Term Drug Efficacy and Biological Stability of Released Drugs

The long-term efficacy and biological stability of drugs released were evaluated through culture of human umbilical vein endothelial cells (HUVECs), which are cells that are involved in angiogenesis. After culturing HUVECs, they were prepared in 2×10⁴ cells/well in a 48-well plate and treated with the long-term released bevacizumab drug samples diluted in 1:1 ratio in a cell culture medium (EBM media containing EGM supplements and 1% P/S) and fresh bevacizumab drug samples by concentration so as to observe cell growth inhibition and safety. For the control of fresh bevacizumab drug samples by concentration, preparation was performed at final concentrations of 1, 10, and 100 g/mL, diluted in cell culture media.

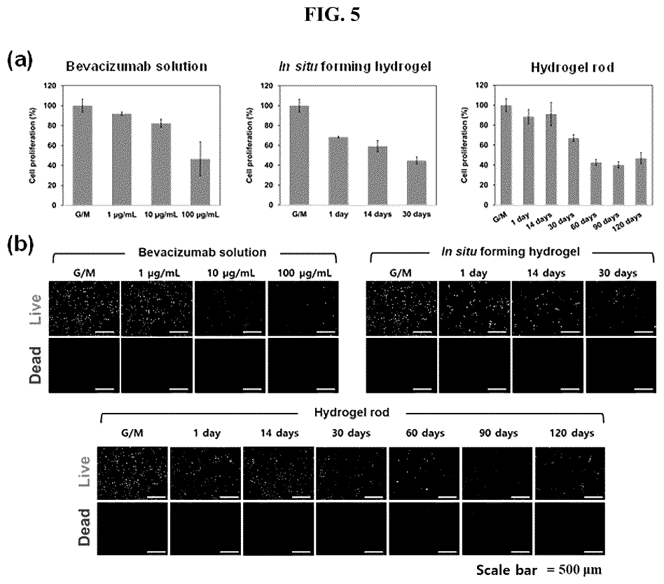

(a) of FIG. 5 shows a result from the WST-1 assay by which inhibition of cell growth in proportion to the concentration of the drug was identified, and an evaluation was conducted on the long-term inhibition of cell growth due to the drug released from the in situ forming hydrogel of Comparative Example and the hydrogel rod of Example. In addition, as shown in (b) of FIG. 5, it was found that there was little cytotoxicity through Live/Dead assay, and cell growth was inhibited compared to the culture cell group through fluorescence images.

Thereby, it is noticeable that the in situ forming hydrogel of Comparative Example shows the rapid release of more than 40% for the initially loaded drug to lead to excessive release, thereby showing the high growth inhibition of cells at the early stage, whereas the hydrogel rod of Example exhibits the drug efficacy through cell growth inhibition for a long period of time because the initial release rate is controlled to exhibit the sustained release for more than four (4) months.

### Experimental Example 4—Animal Experiment and Intraocular Pharmacokinetic Evaluation

### 4-1. Plans for Animal Experiments

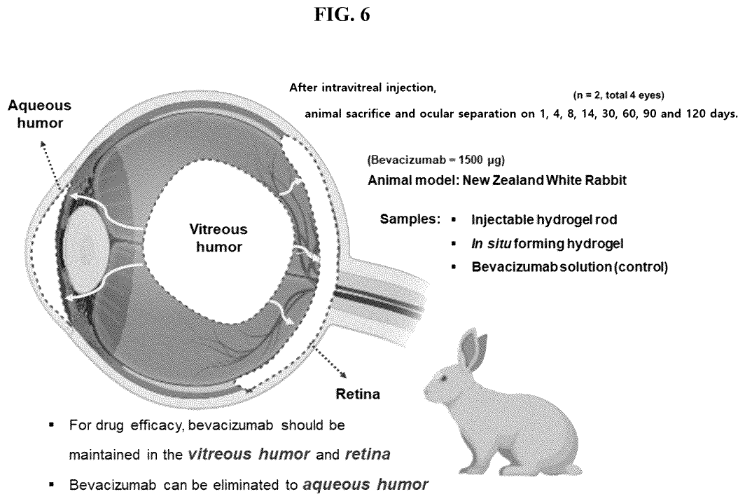

As shown in FIG. 6, intravitreal injections (n=2, total 4 eyes) were carried out using the New Zealand White (NZW) rabbit models. Bevacizumab drug control, hydrogel (in situ forming hydrogel) in Comparative Example, and hydrogel rod in Example were injected. The rabbits were sacrificed on days 1, 4, 8, 14, 30, 60, 90, and 120. In order to maintain its drug efficacy, the concentration of drug must be maintained from vitreous humor to retina, while the drug may be eliminated to the anterior chamber site. For evaluation, pharmacokinetic analysis of vitreous, retina, and aqueous humor tissues was conducted through ELISA after animal sacrifice and ocular separation by date.

### 4-2. Results of Intraocular Pharmacokinetic Evaluation

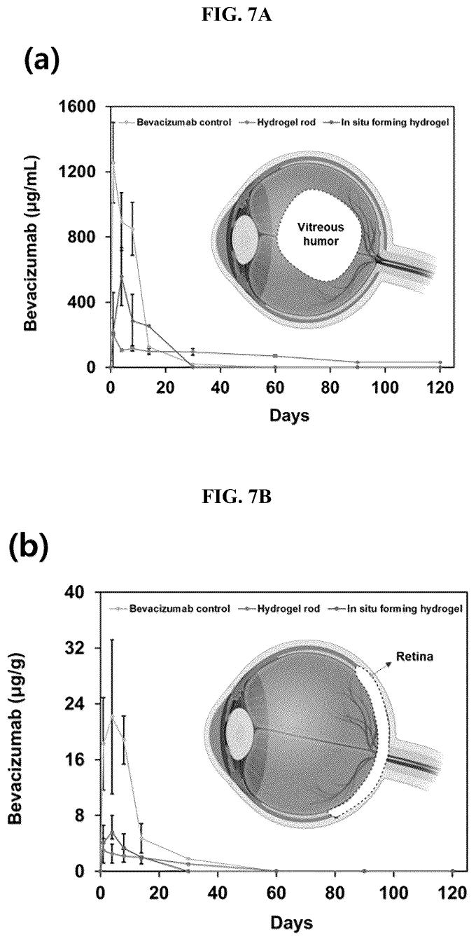

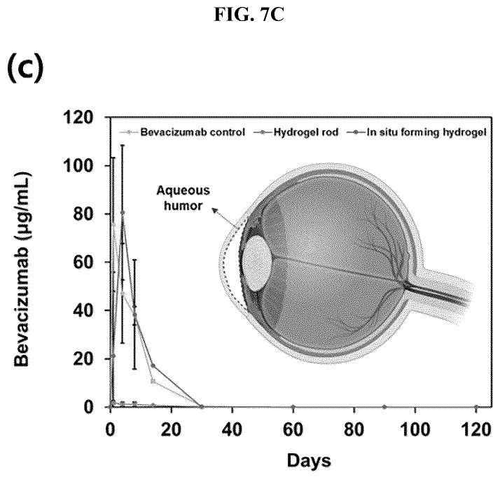

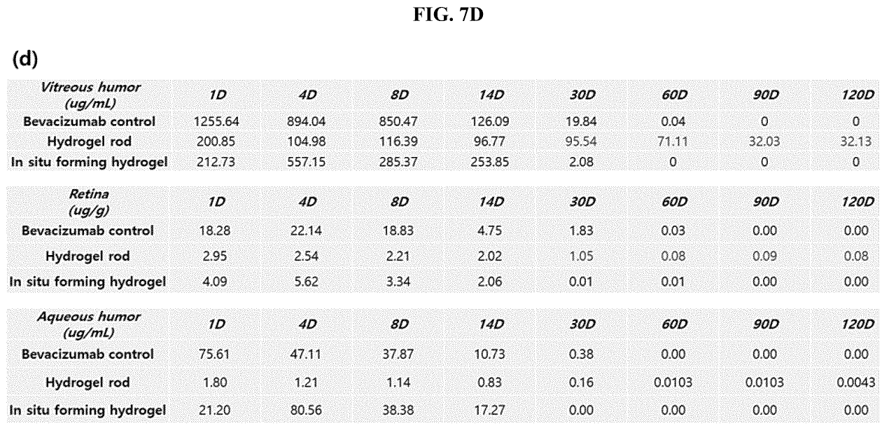

After animal sacrifice and ocular separation by date, pharmacokinetic analysis of vitreous, retina, and aqueous tissues was performed via ELISA. Referring to (a) of FIG. 7, the initial burst release was significantly reduced in the vitreous in the order of control, hydrogel (in situ forming hydrogel) of Comparative Example, and hydrogel rod of Example, and the fastest elimination curve was shown in the control. The initial burst release of hydrogel of Comparative Example decreased by about 2 times compared to the control, but the high release amount was maintained due to dilution with tissue fluid upon injection but did not last for more than 30 days. The hydrogel rod of Example is a pre-crosslinking matrix, and, after the initial burst release is inhibited by 7 times compared to the control and 3 times compared to the hydrogel of Comparative Example, the drug was continuously released within a certain range and the drug concentration of about 32 g/mL was maintained on 120 day, satisfying a level above 1 g/mL which is the drug efficacy threshold of bevacizumab.

Referring to (b) of FIG. 7, as in the retina, the initial burst release was also significantly reduced in the order of control, hydrogel (in situ forming hydrogel) of Comparative Example, and hydrogel rod of Example, and the fastest elimination curve was shown in the control. The hydrogel (in situ forming hydrogel) of Comparative Example, the initial burst release was reduced by about 4 times compared to the control, while the drug concentration was maintained low up to 60 days. The burst release of the hydrogel rod of Example decreased by 10 times compared to the control and about 2 times compared to the hydrogel (in situ forming hydrogel) of Comparative Example, identifying that the drug efficacy may be maintained at a concentration of about 0.08 g/g by sustained release up to 120 days.

Referring to (c) of FIG. 7, the initial excess elimination of the control in a drainway occurred and was maintained until 30 days. The hydrogel (in situ forming hydrogel) of Comparative Example showed an excessive elimination (˜80 g/mL) similar to that of the control from the initial burst release due to phase transition. The hydrogel rod of Example had elimination with reduction by up to approximately 80 times compared to the control and the hydrogel (in situ forming hydrogel) of Comparative Example due to the minimization of the initial burst release caused by pre-crosslinking.

(d) of FIG. 7 shows a quantitative table of drug concentrations for each tissue.

As shown above, it was found that the in situ forming hydrogel of Comparative Example exhibited high initial burst release and elimination to the surrounding tissue, with difficulty in maintaining the drug efficacy for a long time. On the other hand, it was found that the initial burst release was suppressed upon injection of the hydrogel rod of Example to cause sustained release above the concentration for drug efficacy for 4 months, and elimination to the surrounding tissues thereby was greatly reduced. Based on this, it is possible to overcome the issue concerning side effects such as systemic side-effects caused by excessive elimination as well as inflammation caused by frequent injections for drug efficacy.

### Experiment Example 5—Evaluation of Intraocular Conformational Stability and Safety

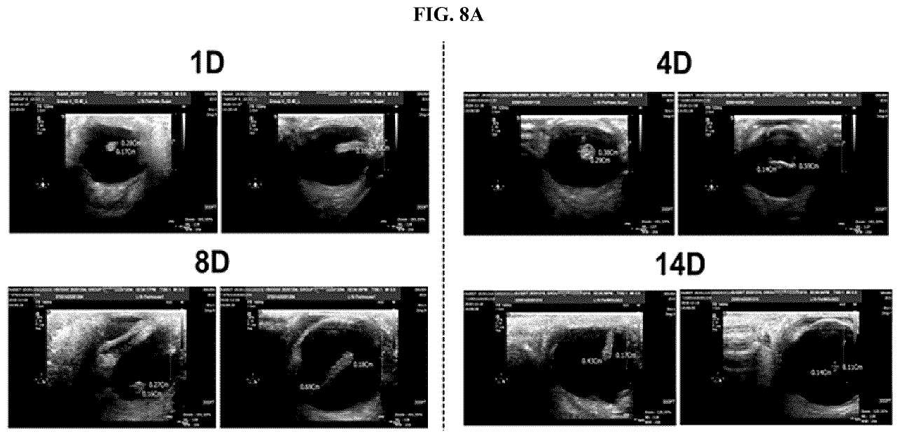

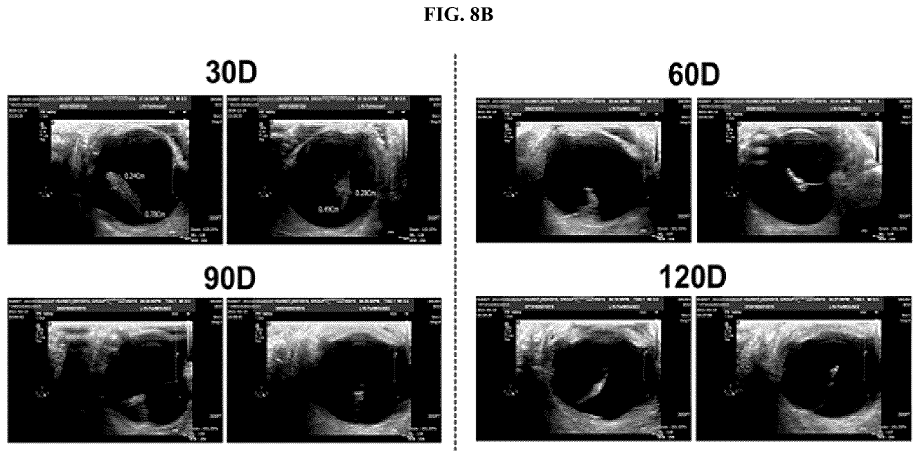

After intraocular injection of the hydrogel rod, ultrasonic analysis was conducted to analyze the conformational stability and safety for each day. As shown in FIG. 8, as a result of imaging by date, it was determined that the conformation of the hydrogel rod remained stable until 120 days, and the size decreased due to degradation in the body from 60 days. No inflammatory reaction or side effect occurred in the body until 120 days, indicating in vivo safety and conformational stability.

It is difficult for the hydrogel of Comparative Example to maintain its shape because it is diluted with the surrounding tissue fluid before the complete phase transition after injection, with a risk of causing side effects through adhesion with the surrounding tissue during the phase transition, but it was found that the hydrogel rod of Example is injected in the form of a crosslinked rod to ensure a stable conformation and safety in vivo for 4 months.

While a specific part of the present disclosure has been described in detail above, it is clear for those skilled in the art that this specific description is merely preferred example embodiments, and the scope of the present disclosure is not limited thereby. In other words, the substantial scope of the present disclosure is defined by the attached claims and their equivalents.

1. An injectable hydrogel rod, comprising:
a rod-shaped hydrogel matrix which comprises a polymer substituted with phenol derivatives and in which the phenol derivatives introduced into side chains of the polymer are bonded to each other and crosslinked; and
a drug loaded in the hydrogel matrix.

2. The injectable hydrogel rod of claim 1, wherein the hydrogel rod exhibits sustained release properties for more than three months by controlling an initial release rate to 10% or less.

3. The injectable hydrogel rod of claim 1, wherein the polymer is one or more polymers selected from the group consisting of gelatin, chitosan, heparin, cellulose, dextran, dextran sulfate, chondroitin sulfate, keratan sulfate, dermatan sulfate, alginate, collagen, albumin, fibronectin, laminin, elastin, vitronectin, hyaluronic acid, fibrinogen, and polybranched-polymers.

4. The injectable hydrogel rod of claim 1, wherein the phenol derivative is one or more selected from the group consisting of hydroxyphenyl propionic acid, 4-hydroxyphenyl acetic acid, tyrosine, tyramine, tetronic tyramine, and PEG-tyramine.

5. The injectable hydrogel rod of claim 1, wherein the drug is one or more drugs selected from intraocularly injectable protein therapeutic agents, chemotherapeutic drugs, low molecular weight therapeutic agents, genes, cells, or cell derivatives.

6. The injectable hydrogel rod of claim 1, wherein hydrogel forms the rod-shaped hydrogel matrix by controlling concentrations of the polymer, an enzyme, and hydrogen peroxide.

7. The injectable hydrogel rod of claim 1, wherein a degree of crosslinking of the hydrogel matrix is 20% to 100%.

8-9. (canceled)

10. An intraocularly injectable implant material comprising the injectable hydrogel rod according to claim 1.

11. A drug delivery carrier comprising the injectable hydrogel rod according to claim 1.

12-19. (canceled)
